SEQUENCE LISTING (7 sequences)

SEQ ID NO: 1 - protein, 8 aa, Homo sapiens
GWGQPHGG

SEQ ID NO: 2 - protein, 8 aa, Homo sapiens
TWIQDNGG

SEQ ID NO: 3 - protein, 8 aa, Homo sapiens
GWIQDQGG

SEQ ID NO: 4 - protein, 8 aa, Homo sapiens
PWIQENGG

SEQ ID NO: 5 - DNA, 762 bp, Homo sapiens
  CDS (1)..(759)
atggcgaaccttggctgctggatgctggttctctttgtggccacatggagtgacctgggcctctgcaagaagcgcccgaagcctggaggatggaacactgggggcagccgatacccggggcagggcagccctggaggcaaccgctacccacctcagggcggtggtggctgggggcagcctcatggtggtggctgggggcagcctcatggtggtggctgggggcagccccatggtggtggctggggacagcctcatggtggtggctggggtcaaggaggtggcacccacagtcagtggaacaagccgagtaagccaaaaaccaacatgaagcacatggctggtgctgcagcagctggggcagtggtggggggccttggcggctacgtgctgggaagtgccatgagcaggcccatcatacatttcggcagtgactatgaggaccgttactatcgtgaaaacatgcaccgttaccccaaccaagtgtactacaggcccatggatgagtacagcaaccagaacaactttgtgcacgactgcgtcaatatcacaatcaagcagcgcacggtcaccacaaccaccaagggggagaacttcaccgagaccgacgttaagatgatggagcgcgtggttgagcagatgtgtatcacccagtacgagagggaatctcaggcctattaccagagaggatcgagcatggtcctcttctcctctccacctgtgatcctcctgatctctttcctcatcttcctgatagtgggatga

SEQ ID NO: 6 - protein, 253 aa, Homo sapiens [sequence omitted]

SEQ ID NO: 7 - protein, 32 aa, Homo sapiens
GWGQPHGGGWGQPHGGGWGQPHGGGWGQPHGG

(19) United States
(12) Patent Application Publication
(10) Pub. No.: US 2004/0053839 A1
(43) Pub. Date: Mar. 18, 2004

(54) Method of protecting cells against apoptosis and assays to identify agents which modulate apoptosis

(76) Inventors: Andrea Leblanc, Chambly (CA); Younes Bounhar, Montreal (CA); Yan Zhang, Montreal (CA)

Correspondence Address: Goudreau Gage Dubuc, Stock Exchange Tower, Suite 3400, PO Box 242 800 Place Victoria, Montreal Quebec, H4Z1E9, CA

(21) Appl. No.: 10/450,679

(22) PCT Filed: Dec. 21, 2001
(86) PCT No.: PCT/CA01/01862

(51) Int. Cl. A61K 38/17

(52) U.S. Cl. 514/12

(57) ABSTRACT

The present invention relates to a method of protecting neurons against bax-mediated apoptosis and assays to identify agents which modulate neuron apoptosis. The present invention further relates to apoptosis modulation in other tissues in which prion protein is expressed, such as heart and lung. The invention further comprises a method of modulating apoptosis in a cell comprising an administration of an apoptosis-modulating effective amount of an agent which interferes with prion protein (PrP)-bax interaction.

### FIELD OF THE INVENTION

The present invention relates to a method of protecting cells against apoptosis and assays to identify agents which modulate apoptosis. More particularly, the present invention relates to a method of protecting neurons against bax-mediated apoptosis and assays to identify agents which modulate neuron apoptosis. The present invention further relates to apoptosis modulation in other tissues in which prion protein is expressed, such as heart and lung. In addition, the present invention relates to a promotion of apoptosis in cells and more particularly in neuron cells.

### BACKGROUND OF THE INVENTION

The role of prion protein (PrP) in prion diseases has been clearly demonstrated over the past 20 years, yet little is known about its function in normal brain. Several functions have been attributed to PrP, however, none of these have been confirmed or linked to prion diseases. Recent work has shown that PrP^{-/-} neuronal cell lines exhibit increased sensitivity to serum deprivation and oxidative stress (1, 2). Moreover, the PrP N-terminal octapeptide repeats share homology to the Bcl-2 homology domain 2 (BH2) (3) and murine PrP interacts with Bcl-2 in the yeast-2-hybrid system (4). Unfortunately, a physiologically relevant role for the murine PrP⁻ Bcl-2 interaction has yet to be provided. There thus remains a need to assess the role of PrP in neuron physiology.

The present invention seeks to meet these and other needs.

The present description refers to a number of documents, the content of which is herein incorporated by reference in their entirety.

### SUMMARY OF THE INVENTION

In one embodiment, the invention concerns a method of modulating apoptosis, and more particularly of protecting apoptosis in tissues and cells. More particularly, such tissues or cells are characterized by enabling prion protein expression. More specifically, the protection of apoptosis is effected by an administration into cells and/or tissues in need of apoptosis protection, of a protection of apoptosis-effective amount of prion protein or part thereof.

The present invention thus further relates to a method of modulating apoptosis and more particularly of protecting against bax-mediated apoptosis in cells, which comprises an administration thereinto of an effective amount of PrP, or part thereof.

The invention in addition relates to a neuroprotection effected by an administration of PrP to neurons.

In addition, the invention relates to a bax-mediated protection in cells in which PrP is expressible, comprising an administration thereinto of an effective amount of PrP. Non-limiting examples of cells or tissues in which PrP is expressible include neurons, cardiac cells and lung cells.

Of note, yeast cells do not naturally express bax. Nevertheless, PrP was shown to be protective of oxidative stress thereinto. Taken together, these data suggest that PrP could have broad apoptosis protection (and modulation) in a number of different cell types.

Further, the present invention relates to an apoptosis-induced stress in a cell comprising an administration of PrP or of an agent in accordance with the present invention.

The Applicant was the first to identify an interaction between PrP and bax or Bcl-2 in vivo, using non-recombinant constructs. Moreover, the Applicant was the first to show that PrP can modulate apoptosis in cells and more particularly in bax-expressing cells.

In accordance with one embodiment of the present invention, the invention concerns the modulation of apoptosis in cells of the CNS. In one particular embodiment, the invention concerns a protective role for PrP in any neurodegenerative disease or condition that involves Bax-mediated cell death. Such disease or condition includes Alzheimer's disease (AD) since it has been demonstrated that Bax increases in AD brains (J. Neuropathol. Exp. Neurol. 1997 56(1):86-93). Other non-limiting examples of diseases or conditions for which PrP could be protective (or could modulate apoptosis) include any disease or condition in which Bax kills cells. Non-limiting examples include Parkinson's, stroke, ischemia, Down Syndrome, multiple system atrophy (i.e. Bax increases in oligodendrocytes), spinal muscular atrophies (SMA), and ALS. Bax could also be implicated in cell death accompanying heart disease such as myocardial infarct.

Of note, in many cancer conditions including leukemia, Bax-increased expression correlates with a good prognosis. Thus, overexpression of prion protein in such cells could lead to inhibition of tumor cell death and thus bad prognosis. Thus, for such applications, the modulation of apoptosis through PrP would have to be opposite to that described above, notably for neuron cell protection. Thus, for such cancer cells, a down-regulation of prion protein activity or level would be beneficial in such a condition. Similarly, it is possible that prion overexpression would favor glioblastoma growth.

Bax-dependent apoptosis is quite common in most cell types and tissues. Non-limiting examples of such cells include neurons, oligodendrocytes, fibroblasts, myoblasts, myotubes, lymphocytes, thymocytes, yeast cells, astrocytes, stem cells, and precursor cells.

It should be understood that apoptosis modulation in accordance with the present invention could be carried-out using numerous approaches. Non-limiting examples thereof include (depending on the direction of apoptosis modulation which is required) antisense technology, competition with peptides, peptidominetics; and regulators of the expression of PrP. It should be understood by the person of ordinary skill that the level, as well as the activity of PrP, will have an impact on the workings of the present invention.

### BRIEF DESCRIPTION OF THE DRAWINGS

Having thus generally described the invention, reference will now be made to the accompanying drawings, showing by way of illustration a preferred embodiment thereof, and in which:

FIG. 1 shows that PrP interacts with Bax and Bcl-2 proteins. A. Alignment of one of four octapeptide repeats at amino acids 56-88 of PrP and the first eight amino acids of the BH2 domain of Bcl-2 related proteins. Conserved amino acids are in bold. Western blots showing co-immunoprecipitation of PrP (3F4) with polyclonal antisera to Bax (pBax, Santa Cruz) in B and immunoprecipitation of Bcl-2 (mBcl-2) with R 55 polyclonal antisera to PrP in C. In B & C, immunoprecipitations of Bax and PrP were confirmed with monoclonal Bax (mBax, Santa Cruz) and PrP (3F4) antibodies. Non-immunoprecipitated protein extracts (40 μg/lane) were used as markers for the immunoprecipitated proteins. D. Autoradiogram of co-immunoprecipitated PrP, Bcl-2 or Bax from in vitro translated (IVT) proteins. Since the Bcl-2 and PrP proteins migrate close together, the first lane of PrP×Bcl-2×Bax contains half the amount PrP×Bcl-2×Bax in order to see the Bcl-2 and PrP co-immunoprecipitation. E. Autoradiogram of IVT PrP and Bax co-imunoprecipitated with PrP R155 antisera (top panel). Western blot analysis of R155-immunoprecipitated PrP with 3F4 PrP antibody and Tau antisera (bottom panel). F. Autoradiogram of IVT PrP and Bax co-immunoprecipitated with pBax antisera. Increasing amounts of PrP to constant amounts of Bax achieve a molar ratio of PrP to Bax of 1.0. G. Co-immunoprecipitation of increasing amounts of IVT Bax and constant amounts of PrP with PrP antisera. Saturation curve and Scatchard plot analysis (inset) show a Kd of 0.44 pM;

FIG. 2 shows that PrP protects against Bax-mediated cell death in yeast and human neurons. A. Survival of yeast transformed with human Bax α cDNA in the absence (▴) or presence of human PrP (□) or Bcl-2 (♦) cDNAs. (▪) and (●) represent PrP and Bcl-2 transformants, respectively. p=0.009 versus Bax only. Yeast S. cerevisiae strain YPH499 was transformed with appropriate vectors (22) and grown in selective HC dropout media. Expression was induced with 2% galactose. Survival was assessed by trypan blue exclusion in samples of 400 cells per experiment. Data represent the mean and standard deviation of three independent transformations. Statistical difference was evaluated with Student t-test. B. Survival of human neurons microinjected with Dextran Texas Red (DTR) and eukaryotic episomal pCep4β constructs expressing human Bax, Bcl-2, PrP or PrPΔOR protein at 0 (first column), 24 (second column), 48 (third column), and 96 (fourth column) hours after injection. PrP×Bax, Bcl-2×Bax, and PrP×Bcl-2×Bax are significantly different from Bax at 24-96 hours (p=0.01). C. Survival of human neurons constructs expressing human PrP, PrP antisense (PrPAS), or Bax at 0 (first column), 12 (second column), or 24 (third column), hours after injection. PrPAS×Bax is significantly different from Bax (p<0.05 at 12 hours and p<0.01 at 24 hours). Neurons were injected as described (23). Each injection was done on 200 neurons in three independent neuronal preparations. Cell death was measured by TUNEL using the in situ cell death detection kit (Boehringer). Eighty to 90% of neurons survive these microinjections and retain DTR for at least 16 days;

FIG. 3 shows the neuroprotection of PrP against oxidative stress independent of Bax. A. Neuroprotection against 10 PM H₂O₂ in neurons microinjected with pCep4β, PrP, PrPΔOR, Bcl-2, and Bax at 0 (solid bar), 24 (white bar) and 48 hours (hatched bar). PrP and Bcl-2 protect against H₂O₂ (p<0.05). B. Yeast cellular survival in 10 μM H₂O₂ in uninduced (black symbol) or induced (clear symbol) Bcl-2 (circle) and PrP (square) transformed yeast. p<0.0002 between induced and uninduced at 102 hours;

FIG. 4 shows the FFI and FASE mutations alter PrP's neuroprotective function against Bax-mediated cell death. A. Apoptosis in neurons microinjected with Cep4β expressing PrP, T183A, D178N in absence or presence of Bax at 0 (first column), 48 (second column) and 96 hours (third column) after microinjection. T183A and D178N significantly reduce PrP's neuroprotective function (p<0.0003). B. Western blot analysis of PrP, Bcl-2 and Bax with 3F4, mBcl-2 and mBax, respectively, in dissociated (diss.) foetal brain cells, primary cultures of human neurons, foetal and adult brains (20 μg/lane). U, M and D represent un-, mono-, and di-glycosylated PrP. C. Western blot analysis of PrP expression in three independent cerebellar protein extracts from foetal and adult brains (20 μg/lane). Proteins from primary neurons, foetal or adult brains were extracted in NP40 lysis buffer (50 mM Tris pH 8.0, 150 mM NaCl, 1% NP40, 5 mM EDTA);

FIG. 5 shows the co-localization of PrP and Bax in neuron cultures. A. Sequential western blot analysis of PrP, Bcl-2, Bax, calnexin (Clnx) and p58 in 10 μg total protein from the subcellular fractionation of the light mitochondrial fraction. B. Immunocytochemical and confocal analysis of Bax (mBax) and PrP (R155). The inset in the Bax and PrP panels represent background staining in absence of primary antibody. C. Confocal analysis of R155 PrP or pBax co-localization with intermediate and cis-Golgi marker p58 antibody, and 3F4 PrP or mBax with polyclonal anti-C₂₁ amyloid precursor protein (APP) antisera; and

FIG. 6 shows sequences of human PrP. A. shows the nucleic acid sequence of PrP. B. shows the amino acid sequence of PrP. C. shows the octapeptide repeat of PrP.

Other objects, advantages and features of the present invention will become more apparent upon reading of the following non-restrictive description of preferred embodiments with reference to the accompanying drawing which is exemplary and should not be interpreted as limiting the scope of the present invention.

### DESCRIPTION OF THE PREFERRED EMBODIMENT

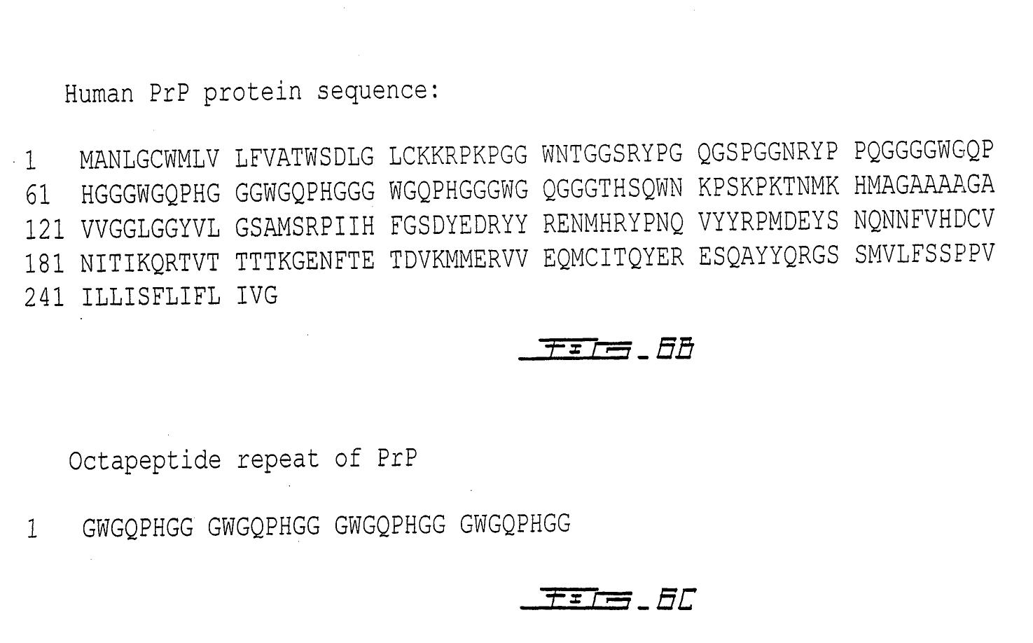

Although the involvement of prion (PrP) protein in several neurodegenerative diseases has long been established, the function of the cellular PrP isoform is still unclear. Here it is demonstrated that PrP interacts and co-localizes with the well-known pro-apoptotic protein Bax. More importantly, PrP abrogates Bax-mediated cell death in yeast and human primary neurons. Deletion of the octapeptide repeat (see FIG. 6) and inherited PrP mutations abolish the neuroprotective function of PrP. It is thus concluded that PrP functions as an anti-apoptotic protein against Bax-mediated cell death in cell in general and in particular in neurons.

### DEFINITIONS

In order to provide a clear and consistent understanding of terms used in the present description, a number of definitions are provided hereinbelow.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA or RNA) for practicing the present invention may be obtained according to well known methods.

As used herein, the term “physiologically relevant” is meant to describe interactions which can modulate transcription of a gene in its natural setting.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised of the deoxyribonucleotides adenine (A), guanine (G), thymine (T) and/or cytosine (C), often in a double-stranded form, which can comprise or include a “regulatory element” as known in the art, as the term is defined herein. The term “oligonucleotide” or “DNA” can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods. While they are usually in a single-stranded form, they can be in a double-stranded form and even contain a “regulatory region”.

As used herein, a “primer” defines an oligonucleotide which is capable of annealing to a target sequence, thereby creating a double stranded region which can serve as an initiation point for DNA synthesis under suitable conditions. Primers can be, for example, designed to be specific for certain variants so as to be used in a variant-specific amplification system.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography . . . ). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain uTATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation.

As used herein, the designation “functional derivative” denotes, in the context of a functional derivative of a sequence whether a nucleic acid or amino acid sequence, a molecule that retains a biological activity (either function or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention.

The functional derivatives of the present invention can be synthesized chemically or produced through recombinant DNA technology. All these methods are well known in the art.

In one particular embodiment, a functional derivative includes a PrP variant which retains its biological activity. One non-limiting example of a PrP variant is a PrP sequence mutagenized in the non-octomer repeat region thereof. While not preferred, a PrP variant could also be mutagenized in the octomer repeat region, provided that the mutation in the octomer repeat region does not destroy the biological activity of the PrP variant.

As used herein, “chemical derivatives” is meant to cover additional chemical moieties not normally part of the subject matter of the invention. Such moieties could affect the physico-chemical characteristic of the derivative (e.g. solubility, absorption, half life, decrease of toxicity and the like). Such moieties are exemplified in Remington's Pharmaceutical Sciences (1980). Methods of coupling these chemical-physical moieties to a polypeptide or nucleic acid sequence are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terminology “PrP polypeptide” or “PrP polynucleotide sequence” refers to a polypeptide or polynucleotide encompassing PrP-derived polypeptides, variant thereof or an active domain thereof or polynucleotides encoding same. Non-limiting examples of PrP polypeptides include polypeptides comprising the amino acid sequence as set forth in FIG. 6C, FIG. 6B, and variants or fragments thereof. As used herein, the term “active domain of PrP”, “biologically active polypeptide of PrP” or the like refers to a polypeptide fragment or portion of PrP and more particularly human PrP (although not limited to human PrP) that retains an activity of PrP. The term “PrP polypeptide” is also meant to encompass PrP or an active domain thereof that is fused to another polypeptide, such as a non-PrP polypeptide sequence.

“PrP activity” “polypeptide comprising the amino acid sequence set forth in FIG. 6C or variants thereof retaining PrP activity” or “biological activity” of PrP or other polypeptides of the present invention is defined as a detectable biological activity of a PrP gene, PrP nucleic acid sequence, PrP protein or PrP polypeptide of the present invention. This includes any physiological function attributable to the specific biological activity of PrP, or of factors a PrP sequence interacts with. This includes measurement of the neuroprotective activity of PrP in cells, in animals or in vitro. Non-limiting examples of the biological activities may be made directly or indirectly. PrP biological activity is not limited, however, to its function in neuroprotection. Biological activities may also include simple binding to other factors (polypeptides or otherwise), including compounds, substrates, and of course interacting proteins (e.g. Bax or Bcl-2). Thus, for PrP, biological activity includes any standard biochemical measurement of PrP such as conformational changes, phosphorylation status or any other feature of the protein that can be measured with techniques known in the art. PrP biological activity also includes activities related to PrP gene transcription or translation, or any biological activities of such transcripts or translation products. The instant invention is also concerned with PrP interaction with interacting polypeptide of the present invention, biological activity of PrP and fragment thereof also includes assays which monitor binding and other biochemical measurements of these polypeptides. Furthermore, for certainty, the terminology “biological activity” also includes measurements based on the interaction of domains of interacting proteins of the present invention.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a defect in neuroprotection, apoptosis modulation, oxydative stress, cancer, neurodegenerative diseases and the like. Alternatively, the molecules identified in accordance with the teachings of the present invention find utility in the development of more efficient modulators of neuroprotection, bax-mediated apoptosis or oxydative stress.

As used herein, agonists and antagonists of PrP-Bax or PrP-Bcl-2 interaction can be detected by contacting an indicator cell with a compound or mixture or library of molecules for a fixed period of time and the interaction is then determined.

Of course, such modulators of the interaction could be identified by assaying for another biological function of PrP or variant or fragment thereof.

It shall be understood that the “in vivo” experimental model can also be used to carry out an “in vitro” assay. For example, cellular extracts from the indicator cells can be prepared and used in one of the “in vitro” tests of the present invention or others well-known in the art.

As used herein the recitation “indicator cells” refers to cells that express a PrP polypeptide or PrP polynucleotide of the present invention. In another embodiment, the indicator cell also expresses an interacting factor of PRP (e.g. Bax, Bcl-2) and wherein an interaction between PrP and the interacting factor or domains thereof is coupled to an identifiable or selectable phenotype or characteristic such that it provides an assessment of the interaction between same. Such indicator cells can be used in the screening assays of the present invention. In certain embodiments, the indicator cells have been engineered so as to express a chosen derivative, fragment, homolog, or mutant of PrP interacting factor. The cells can be yeast cells or higher eukaryotic cells such as mammalian cells (WO 96/41169). Preferably, the indicator cells are yeast cells. In one particular embodiment, the indicator cell is a yeast cell harboring vectors enabling the use of the two hybrid system technology, as well known in the art (Ausubel et al., 1994, supra) and can be used to test a compound or a library thereof. In one embodiment, a reporter gene encoding a selectable marker or an assayable protein can be operably linked to a control element such that expression of the selectable marker or assayable protein is dependent on the interaction of the domains of PrP and Bax. Such an indicator cell could be used to rapidly screen at high-throughput a vast array of test molecules. In a particular embodiment, the reporter gene is luciferase or β-Gal.

In one embodiment, at least one of the interaction domain of the present invention may be provided as a fusion protein. The design of constructs therefor and the expression and production of fusion proteins are well known in the art (Sambrook et al., 1989, supra; and Ausubel et al., 1994, supra). In another embodiment, both interaction domains are part of fusion proteins. In one particular embodiment, the fusions are a LexA-PrP polypeptide fusion (DNA-binding domain—PrP polypeptide; bait) and a B42-Bax polypeptide fusion (transactivator domain-Bax polypeptide; prey). In one such embodiment, the LexA-PrP and B42-Bax fusion proteins are expressed in a yeast cell also harboring a reporter gene operably linked to a LexA operator and/or LexA responsive element.

Non limiting examples of such fusion proteins include hemaglutinin fusions and Gluthione-S-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. In certain embodiments, it might also be beneficial to introduce a linker (commonly known) between the interaction domain and the heterologous polypeptide portion. Such fusion protein find utility in the assays of the present invention as well as for purification purposes, detection purposes and the like.

For certainty, the sequences and polypeptides useful to practice the invention include without being limited thereto mutants, homologs, subtypes, alleles and the like. It shall be understood that generally, the sequences of the present invention should encode a functional (albeit defective) interaction domain. It will be clear to the person of ordinary skill that whether an interaction domain of the present invention, variant, derivative, or fragment thereof retains its function in binding to its partner can be readily determined by using the teachings and assays of the present invention and the general teachings of the art.

As exemplified herein below, the interaction domains of the present invention can be modified, for example by in vitro mutagenesis, to dissect the structure-function relationship thereof and permit a better design and identification of modulating compounds. However, some derivative or analogs having lost their biological function of interacting with their respective interaction partner (Bax or Bcl-2) may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of PrP-Bax interaction for example.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, such an advantage might be rendered moot if both polypeptide tested directly interact. It will be understood that extracts from mammalian cells for example could be used in certain embodiments, to compensate for the lack of certain factors.

As exemplified herein, the present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present invention can be introduced into individuals in a number of ways. For example, erythropoietic cells can be isolated from the afflicted individual, transformed with a DNA construct according to the invention and reintroduced to the afflicted individual in a number of ways, including intravenous injection. Alternatively, the DNA construct can be administered directly to the afflicted individual, for example, by injection in the bone marrow. The DNA construct can also be delivered through a vehicle such as a liposome, which can be designed to be targeted to a specific cell type, and engineered to be administered through different routes.

For administration to humans, the prescribing medical professional will ultimately determine the appropriate form and dosage for a given patient, and this can be expected to vary according to the chosen therapeutic regimen (e.g. DNA construct, protein, cells), the response and condition of the patient as well as the severity of the disease.

Composition within the scope of the present invention should contain the active agent (e.g. fusion protein, nucleic acid, and molecule) in an amount effective to achieve the desired therapeutic effect while avoiding adverse side effects. For example, a neuroprotective domain of PrP. Typically, the nucleic acids or polypeptide (or compound) in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th Ed., Mack Ed.). For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal.

Further, the invention encompasses a process for producing a pharmaceutical composition comprising: a) carrying out a screening assay of the present invention aimed at identifying a compound that is active on a PrP polypeptide or biologically active fragment or variant thereof, wherein the screening assay enables the identification of a candidate compound as a compound that is active on a PrP polypeptide; and b) mixing the compound identified in a) with a suitable pharmaceutical carrier. In one embodiment, the PrP polypeptide comprises the amino acid sequence as set forth in FIG. 6C or biologically active fragment or variant thereof. In one particular embodiment, the compound is a neuroprotective compound. In an alternative embodiment, the compound is an apoptosis inducing compound.

In a further embodiment of this process of producing a pharmaceutical composition, the process further includes a scaling-up of the preparation for isolating of the identified compound active on the PrP polypeptide. In yet another embodiment of this process of producing a pharmaceutical composition, the pharmaceutical composition prepared comprises a derivative or homolog of the compound identified in a).

The present invention relates to a kit for diagnosing a neurodegenerative disease or condition or a predisposition to contracting same comprising a nucleic acid, a protein or a ligand in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (DNA protein or cells), a container which contains the primers used in the assay, containers which contain enzymes, containers which contain wash reagents, and containers which contain the reagents used to detect the extension products.

The present invention also relates to a kit comprising the oligonucleotide primer of the present invention, which are specific to PrP.

Yet in another embodiment, the present invention relates to an assay to screen for drugs for the treatment and/or prevention of neurodegenerative diseases, Bax-mediated cell death-associated diseases and cancer. In a particular embodiment, such assays can be designed using cells from patients having a genotype known or suspected of being associated with such a disease or condition. These cells harboring recombinant vectors can enable an assessment of the functionality of a PrP sequence. Non-limiting examples of assays that could be used in accordance with the present invention include cis-trans assays similar to those described in U.S. Pat. No. 4,981,784.

In accordance with the present invention, there is also provided a method for identifying, from a library of compounds, a compound with therapeutic effect on one of the diseases or conditions of the present invention comprising providing a screening assay comprising a measurable biological activity of PrP protein or gene; contacting the screening assay with a test compound; and detecting if the test compound modulates the biological activity of PrP protein or gene; wherein a test compound which modulates the biological activity is a compound with this therapeutic effect.

While human PrP are preferred sequences (nucleic acid and proteins) in accordance with the present invention, the invention should not be so limited. Indeed, in view of the significant conservation of the PrP sequences throughout evolution, sequences from different species, and preferably mammalian species, could be used in the assays of the present invention.

Generally, high throughput screens for PrP modulators of apoptotic/proliferation function, i.e. candidate or test compounds or agents (e.g., peptides, peptidomimetics, small molecules or other drugs) may be based on assays which measure biological activity of PrP. The invention therefore provides a method (also referred to herein as a “screening assay”) for identifying modulators, which have a stimulatory or inhibitory effect on, for example, PrP biological activity or expression, or which bind to or interact with PrP proteins, or which have a stimulatory or inhibitory effect on, for example, the expression or activity of PrP interacting proteins (targets) or substrates.

In one embodiment, the invention provides assays for screening candidate or test compounds which interact with substrates of a PrP protein or biologically active portion thereof.

In another embodiment, the invention provides assays for screening candidate or test compounds which bind to or modulate the activity of a PrP protein or polypeptide or biologically active portion thereof.

The assays described herein may be used as initial or primary screens to detect promising lead compounds for further development. The same assays may also be used in a secondary screening assay to measure the activity of candidate compounds on a PrP polypeptide (or polynucleotide). Often, lead compounds will be further assessed in additional, different screens. This invention also includes secondary PrP screens which may involve biological assays utilizing specific cell types.

Tertiary screens may involve the study of the effect of the agent in an animal. Accordingly, it is within the scope of this invention to further use an agent identified as described herein in an appropriate animal model. For example, a test compound identified as described herein (e.g., PrP sequence, an antisense PrP nucleic acid molecule, a PrP-specific antibody, or a PrP-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above described screening assays for treatment (e.g. bacterial infections), as described herein.

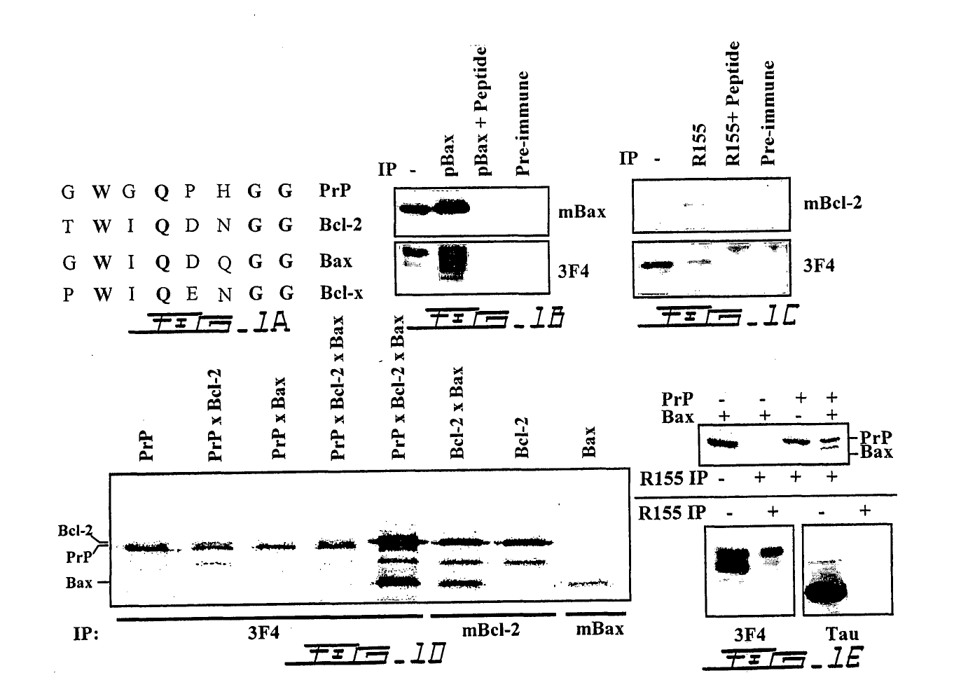

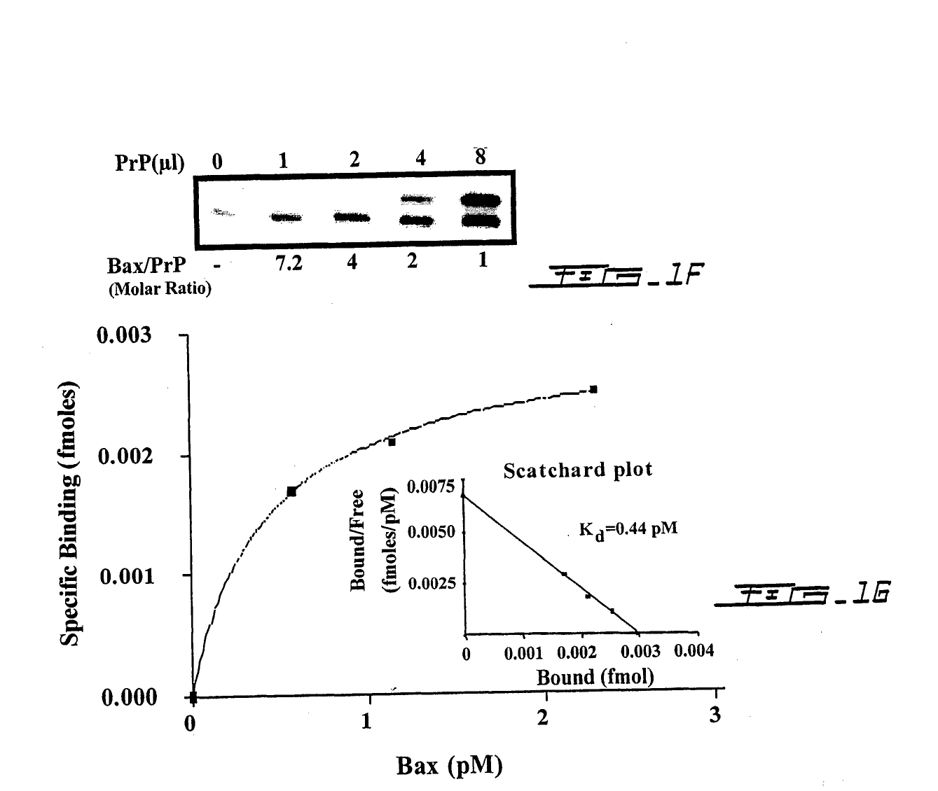

Bcl-2 family members share BH domains known to mediate function sometimes through homo- and hetero-dimerization (5-7). There is 50% homology between the first eight amino acids of the BH2 domain of Bcl-2 proteins and four octapeptide repeats in the N-terminal region of PrP (FIG. 1A). The homology between BH2 and PrP indicates that PrP may be a member of the Bcl-2 family and may play a role in neuronal survival by interaction with Bcl-2 family members.

The interaction of PrP with Bax or Bcl-2 by co-immunoprecipitations from foetal brain protein extracts was thus assessed. Immunoprecipitations from brain tissue were carried out with 150 μg of NP-40 soluble proteins, 1% protein A agarose, 1× modified RIPA buffer (10 mM sodium phosphate, 0.15M sodium chloride, 1% NP-40, 2 mM EDTA pH 8, 0.05% PMSF, 0.1 μg/ml pepstatin A, 1 μg/ml TLCK, 0.5 μg/ml leupeptin) and 1/100 dilutions of either anti-human Bcl-2, PrP, or Bax antisera. PrP co-immunoprecipitates with Bax protein using either Bax or PrP antsera (FIGS. 1B & 1E). In addition, PrP R155 polyclonal antisera (raised against residues 36-56) co-immunoprecipitates Bcl-2 (FIG. 1C). Co-immunoprecipitation of PrP with Bax or Bcl-2 is abolished by competitions with the appropriate synthetic antigenic peptide (1 μg) or is absent with pre-immune sera immunoprecipitations (FIGS. 1B & C). Cytoskeletal Tau does not co-immunoprecipitate with PrP antisera (FIG. 1E). These results show that PrP interacts with Bcl-2 and Bax. To determine if PrP interacts independently with Bax or Bcl-2 monomeric proteins or interacts with a Bcl-2-Bax heterodimer, equimolar amounts of in vitro translated (IVT) PrP, Bax and Bcl-2 (Example 1) were mixed and immunoprecipitated with PrP in 1×modified RIPA containing 30 μl protein-A-agarose, 2 μl antibody (pBax or R155) and IVT proteins in a total volume of 100 μl. (FIG. 1D). PrP co-immunoprecipitates Bcl-2 or Bax from PrP×Bcl-2 and PrP×Bax mixes, respectively, and Bcl-2 and Bax from PrP×Bcl-2×Bax mixes of proteins. Bax was shown co-immunoprecipitate with Bcl-2. These results confirm the direct interaction of PrP with Bcl-2 or Bax. PrP co-immunoprecipitation with Bax (FIG. 1F) and Bax co-immunoprecipitation with PrP are saturable and binding affinity was calculated at Kd=0.44 pM (FIG. 1G; Example 1). Taken together, these results open the way to assays and methods to identify apoptosis modulators (e.g. two hybrid assays, binding assays and the like). Based on these observations, it was postulated that PrP might protect against Bax-mediated cell death.

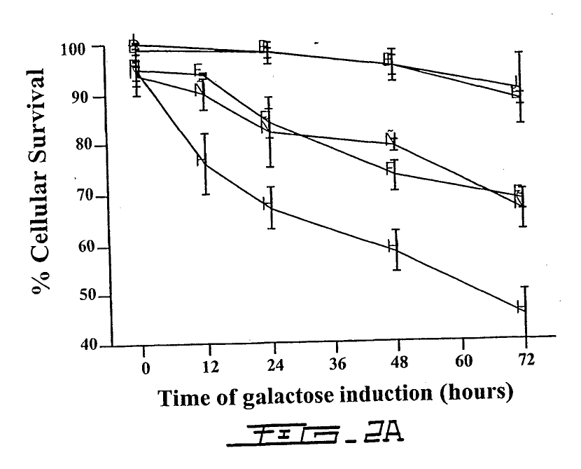

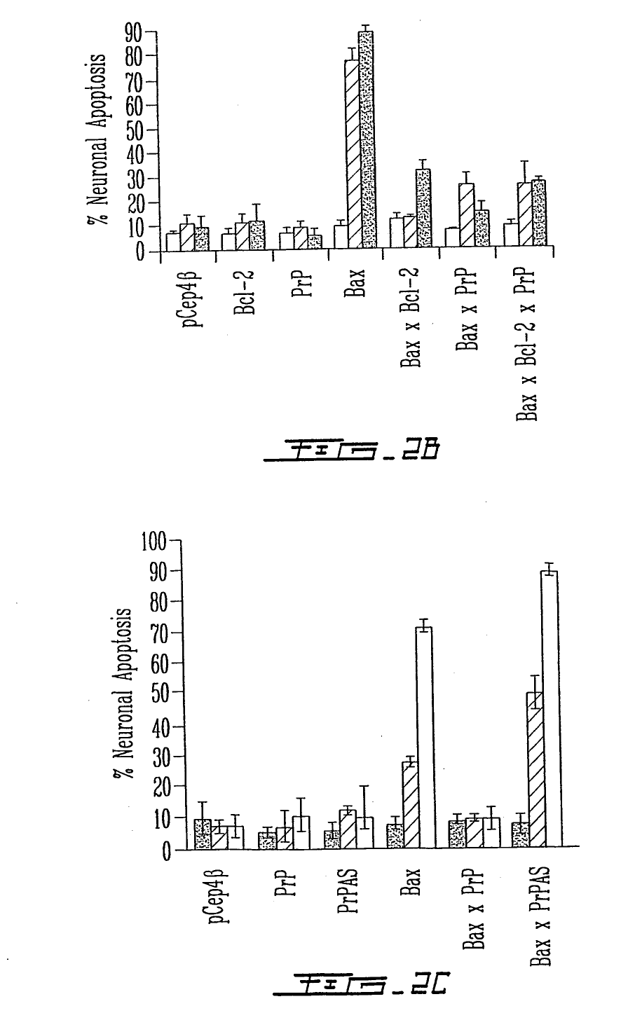

Bax expression in yeast induces cell death, rescuable by Bcl-2 (9). To assess the function of PrP against Bax-mediated cell death, yeast were transformed with the galactose-inducible p42X-Gal1 episomal constructs expressing human Bax, PrP or Bcl-2. The induction of Bax expression in yeast transformants results in 50% cell death within 72 hours (FIG. 2A). Co-transformation of the Bax cell line with either PrP or Bcl-2 protects against Bax-mediated cell death. Uninduced co-transformants (not shown) or Bcl-2 and PrP yeast transformants all survive. These results show that PrP protects against Bax-mediated cell death.

Prion diseases target the nervous system exclusively. The role of PrP against Bax-mediated cell death in human primary neurons in culture was therefore assessed. cDNA was directly microinjected in neurons. Injection of Bax cDNA, but not vector pCep4β, Bcl-2 or PrP cDNA induces a rapid cell death in 90% of neurons within 48 hours (FIG. 2B). Co-injection of either PrP or Bcl-2 with Bax protects against Bax-mediated cell death. The triple-injection of PrP and Bcl-2 with Bax does not further enhance protection, suggesting that PrP and Bcl-2 act through the same mechanism. To determine the importance of the octapeptide repeat, the neuroprotective ability of a PrP mutant lacking the octapeptide repeat region (PrPΔOR) was tested. Interestingly, the deletion of the octapeptide repeats abolishes the neuroprotective function of PrP (FIG. 2B). Endogenous PrP also is neuroprotective since PrP antisense expression increases the rate of Bax-mediated neuronal cell death (FIG. 2C). Therefore, these results confirm PrP's neuroprotective function against Bax-mediated cell death and indicate that the octapeptide repeat region of PrP is necessary for function. These observations may explain the mechanism by which PrP ablation increases cell vulnerability (1, 2). These results also show another embodiment of the present invention: a promotion of apoptosis through a decrease in PrP protection (e.g. PrP antisense expression).

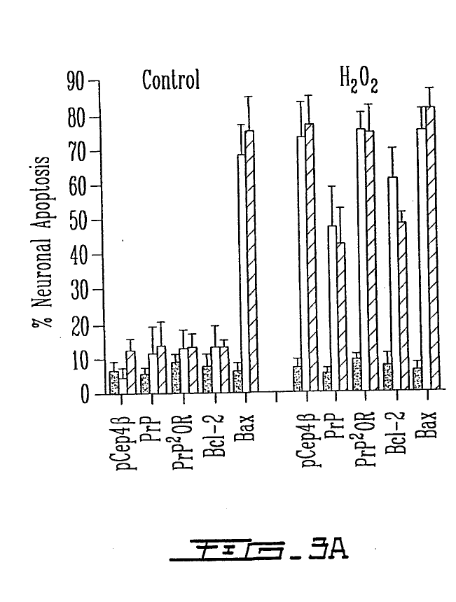

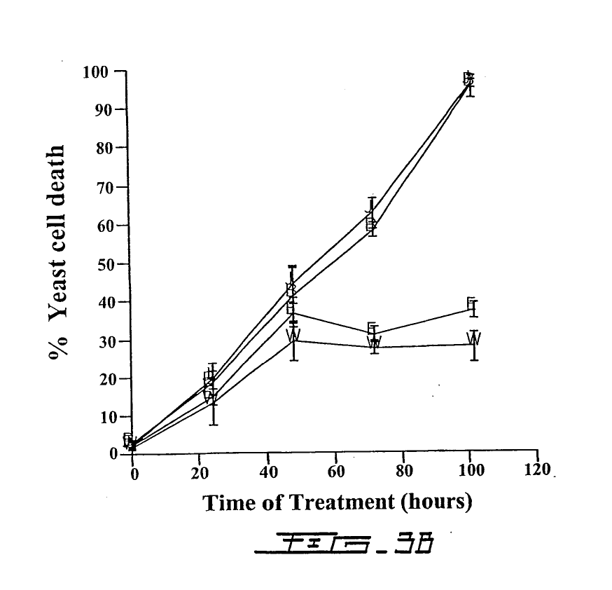

PrP is also neuroprotective against oxidative stress through the octapeptide repeat (2, 10). PrP and Bcl-2 but not PrPΔOR partially protect neurons against 10 μM H₂O₂ was confirmed (FIG. 3A). However, while PrP protects almost entirely against Bax-mediated cell death, it only prevents 30% neuronal apoptosis with H₂O₂₋. Knowing that oxidative stress can increase Bax expression (11), the efficiency of PrP protection against oxidative stress was tested and shown to be Bax-independent in Bax-deficient yeast cells (FIG. 3B). Again, similar to Bcl-2, PrP can protect yeast cells against oxidative stress. These results indicate that PrP can be protective through two alternative mechanisms.

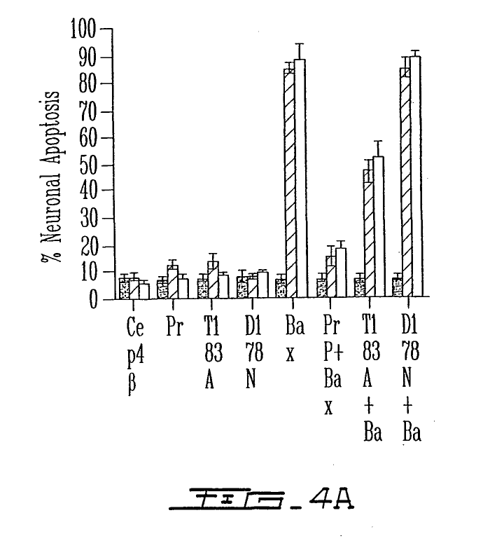

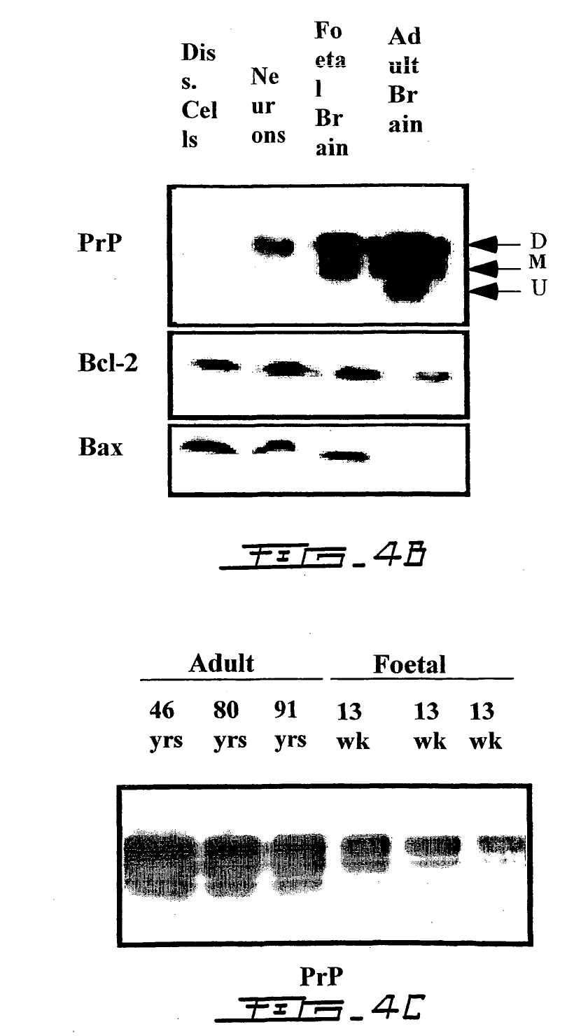

Mutations of PrP associated with prion diseases to assess whether they could alter the neuroprotective ability of PrP against Bax-mediated cell death (FIG. 4A). While the T183A Familial Atypical Spongiform Encephalopathy (FASE) mutation (12) partially inhibits PrP's function, the D178N Fatal Familial Insomnia (FFI) mutation (13) completely abolishes PrP's neuroprotective function against Bax. These results support the hypothesis that inherited PrP mutations alter PrP's neuroprotective function. According to this model, mutant PrP should lead to neurodegeneration in the developing CNS since Bax is a predominant pathway for neuronal apoptosis (14). Yet, prion diseases only occur later in life. To explore this apparent paradox, the pattern of PrP, Bcl-2 and Bax expression in human foetal and adult brains was examined. PrP levels are considerably higher in mature brain compared to foetal brain or neurons in culture (FIG. 4B). In contrast, Bcl-2 and Bax levels are relatively high in foetal brains and neurons and decrease with age. Comparison of PrP levels in foetal and adult cerebella shows that the shift in PrP levels is a common feature of humans (FIG. 4C). One can thus speculate that PrP may be more important in the aging brain, and that mutant PrP cannot compensate for the decreasing Bcl-2 levels with age. In any event, the results presented herein also provide the basis for a diagnosis and prognosis assay for neurodegenerator in the CNS. One example is a diagnostic assay which identifies mutations in PrP in CNS, which have the potential to significantly affect neuronal cell survival.

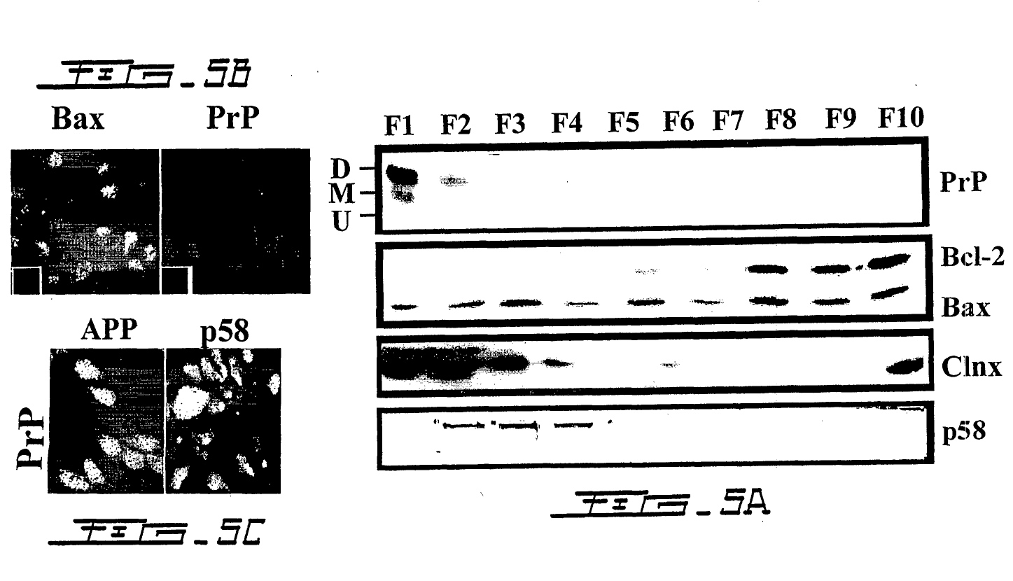

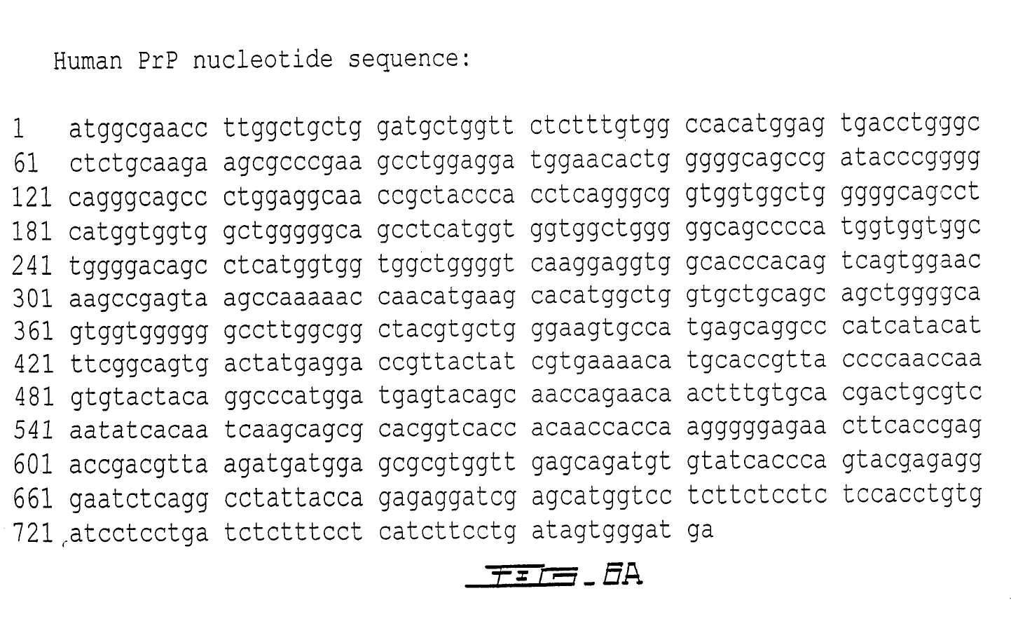

The T183A mutant PrP is strictly intracellular (15, 16). However, T183A PrP is still neuroprotective indicating that an intracellular transmembrane form of PrP, such as previously described (17, 18) mediates function (FIG. 4A). Fractionation of the light mitochondrial fraction of neurons (Example 2) shows the presence of Bax and mono- and di-glycosylated forms of PrP in the expected Golgi fractions confirmed by p58 immunostaining (FIG. 5A; F1 to F4). T183A PrP is thus another example of a PrP variant maintaining its biological activity, while D178N is an example of a variant which loses its neuroprotective biological function. The present invention thus provides the means to assess the functionality of a variant PrP. Although calnexin is an endoplasmic reticulum-specific marker in most cells, it is detected also in the intermediate compartment/cis-Golgi in neurons (20). In addition, Bcl-2, Bax and non- and mono-glycosylated PrP are detected in the expected endoplasmic reticulum fractions (F9 and F10). Since subcellular fractionations cannot exclude the presence of small amounts of contaminating plasma membrane GPI-anchored PrP, the co-localization of PrP with Bax was confirmed by confocal immunofluorescence (FIG. 5B). The neuron cultures were treated with PIPLC to remove GPI-anchored proteins before immunostaining (Example 3). PrP does not localize to the cell surface or significantly to the cis-Golgi in neurons as demonstrated by PrP co-staining with APP or cis-Golgi p58 marker (FIG. 5C). However, in astrocytes, PrP staining strongly co-localizes with p58. While Bax also does not co-localize with APP, it strongly co-localizes with p58 in some neurons. These results are consistent with the cytosolic part of a neuronal transmembrane form of PrP present in a trans-Golgi compartment being co-localized with cytosolic Bax.

The present invention thus shows that PrP possesses a Bax-independent protection activity. In addition, PrP protects against Bax-mediated neuronal apoptosis. Inherited mutations of PrP alter this function, suggesting that familial prion diseases may be due to a loss of the neuroprotective PrP function. The role of Bax in the apoptosis of human CNS neurons was also confirmed. In addition, it was herein demonstrated that PrP is homologous to and interacts with Bax protein, and inhibits Bax-mediated apoptosis, making it likely to be a new and unexpected member of the ever growing Bcl-2 family of proteins. Taken together, these findings are directly relevant to uncovering molecular mechanisms of survival and apoptosis in normal and neurodegenerating human CNS neurons. The present invention thus opens the way to diagnosis, prognosis and therapeutic approaches to neurodegenerative diseases. It also opens the way to the design of numerous types of assays to screen and identify agents which could mimic the PrP apoptosis modulation mechanism. In one embodiment, the invention relates to assays based on a PrP-bax interaction.

The present invention is illustrated in further detail by the following non-limiting examples.

### EXAMPLE 1

### Synthesis of PrP, Bcl-2 and Bax

PrP, Bcl-2 and Bax were synthesized using coupled in vitro transcription/translation TnT-7 system (Promega). Proteins are of the expected size and immunoprecipitated with the appropriate antibodies. PrP and Bcl-2 show a lower MW protein that could represent an early termination, a downstream AUG initiation or a proteolytic fragment occurring during translation. For the quantitative binding assay, the amount of protein immunoprecipitated was determined by phosphorimaging and calculated based on an ³⁵S-methionine standard curve taking into account the number of methionine residues per PrP or Bax molecule.

### EXAMPLE 2

### Subcellular Fractionation Studies

Subcellular fractionations were done at 4° C. with Optiprep self-forming gradients according to the manufacturer's instructions (Pharmacia. The cells were homogenized in diluent B (8% sucrose, 1 mM EDTA, 20 mM Tricine/NaOH pH 7.8) and centrifuged at 1000 g for 10 minutes. The supernatant (S) was centrifuged consecutively at 3000 g, 10 minutes for P2 and S2, at 17,500 g, for 15 minutes for P3 and S3. The P3 light mitochondrial fraction was resuspended in diluent B and 17.5% Optiprep and centrifuged at 270,000 g for 3 hours. Equal volumes of each fraction were collected from top to bottom to yield F1 to F10, respectively. For protein analysis, fractions were solubilized in sample buffer.

### EXAMPLE 3

### Immunostaining of Neurons

Neurons were treated with 300 units/ml of phosphatidyl inositol phospholipase C (PIPLC; Novato, Calif.) 45 minutes at 37° C. Released PrP was confirmed by immunoprecipitation from radiolabeled conditioned media (data not shown). Neurons were fixed in fresh 4% paraformaldehyde and 4% sucrose 20 minutes, permeabilized in 0.25% Triton X-100 for 10 minutes, blocked in 10% fetal goat serum 20 minutes, and incubated with 1/50 dilution of the primary antibodies. Detection of the primary antibodies was done with anti-mouse or rabbit immunoglobulin conjugated to fluorescein or rhodamine, respectively. The Bio-Rad MRC600 was used for confocal analysis.

Although the present invention has been described hereinabove by way of preferred embodiments thereof, it can be modified without departing from the spirit and nature of the subject invention as defined in the appended claims.

### REFERENCES

1. Kuwahara et al., Nature 400, 225 (1999).

2. Brown et al., Exp. Neurol. 146, 104 (1997).

3. LeBlanc A., in Handbook of the Aging Brain. S. S. a. E. Wang, Eds. (Academic Press, NY, 1998) pp. 202.

4. Kurschner et al., Mol. Br. Res. 30, 165 (1995).

5. Adams et al., Science 281, 1322 (1998).

6. Yin et al., Nature 369, 321 (1994).

7. Oltvai et al., Cell 74, 609 (1993).

9. Matsuyama et al., Mol. Cell 1, 327 (1998).

10. Brown et al., Biochem. J. 344, 1 (1999).

11. Riou et al., Endocrinol. 140, 1990 (1999).

12. Nitrini et al., Ann. Neurol. 42, 138 (1997).

13. Medori et al., N Engl J Med 326, 444 (1992).

14. Putcha et al., J. Neurosci. 19, 7476 (1999).

15. Rogers et al., Glycobiol. 1, 101 (1990).

16. DeArmond et al., Neuron 19, 1337 (1997).

17. Hegde et al., Science 279, 827 (1998).

18. Hegde et al., Nature 402, 822 (1999).

20. Krijnse-Locker et al., Mol. Biol. Cell 6, 1315 (1995).

22. Gietz et al., Methods Mol. Cell. Biol. 5, 255 (1995).

23. Zhang et al., J. Neurosci. 20, in press (2000).

What is claimed is:

1. A method of modulating apoptosis in a cell comprising an administration of an apoptosis-modulating effective amount of an agent which interferes with prion protein (PrP)-bax interaction.

2. Method of protecting a cell against apoptosis comprising an administration thereto of an apoptosis-modulating effective amount of PrP or fragment thereof, said fragment retaining said apoptosis protection activity.

3. The method of claim 2, wherein said apoptosis is bax-induced.

4. The method of claim 2 or 3, wherein said fragment comprises the octapeptide repeat of PrP.

5. The method of claim 1, 2, 3 or 4, wherein said cell is a cell from the central nervous system.

6. The method of claim 5, wherein said cell is a neuron.

7. Use of PrP to protect against bax-mediated apoptosis in cells in which PrP is expressible, comprising an administration thereinto of an effective amount of PrP or biologically active fragment or variant thereof.

8. The use of claim 7, wherein said cell is selected from neurons, oligodendrocytes, fibroblasts, myoblasts, myotubes, lymphocytes, thymocytes, yeast cells, astrocytes, stem cells, and precursor cells.

9. An assay for screening and selecting an agent which modulates bax-dependent apoptosis comprising:
contacting i) a recombinant prion protein PrP or functional fragment thereof, which comprises the octapeptide repeat; and ii) a recombinant bax protein or part thereof which directly interacts with said PrP or fragment thereof; and
assaying an interaction between i) and ii) wherein an agent which modulates said apoptosis is selected when the interaction between i) and ii), in the presence of said agent, is significantly different than in the absence thereof.

10. The assay of claim 9, wherein at least one of i) and ii) is a fusion protein expressible from a fusion protein harbored by an expression vector expressing a fusion protein comprising PrP or a fragment thereof, said fragment comprising a functional octapeptide repeat of PrP; or a second expression vector expressing a second fusion protein comprising bax or a fragment thereof.

11. The assay of claim 10, being a two-hybrid system assay.

12. The assay of claim 9, being a cis-trans assay.

13. A method to diagnose or prognose apoptosis in a cell comprising an assessment of the apoptotic-protection activity of PrP expressed therein.
